(19) United States
(12) Patent Application Publication
(10) Pub. No.: US 2024/0374502 A1
(43) Pub. Date: Nov. 14, 2024

(54) TOOTHPOWDER COMPOSITION

(71) Applicant: DR. TUNG'S PRODUCTS, INCORPORATED, KANEOHE, HI (US)

(72) Inventor: Colin DAVIS, Kaneohe, HI (US)

(73) Assignee: DR. TUNG'S PRODUCTS, INCORPORATED, KANEOHE, HI (US)

(21) Appl. No.: 18/649,039

(22) Filed: Apr. 29, 2024

Related U.S. Application Data
(60) Provisional application No. 63/465,420, filed on May 10, 2023.

(51) Int. Cl. A61K 8/9783; A61Q 11/00

(52) U.S. Cl. CPC A61K 8/9783; A61Q 11/00; A61K 2800/92

(57) ABSTRACT

An oral treatment composition and methods of use for teeth cleaning and treating dry mouth. The oral treatment composition includes effective amounts of organic ingredients including neem, miswak, and triphala, embodied as a mouth chewable tablet. The composition includes one or more of the following ingredients: xylitol, bentonite, calcium carbonate, sodium bicarbonate, Salvadora persica stem, Cyamopsis tetragonoloba (guar) gum, Eugenia caryophyllus (clove), Cymbopogon flexuosus (lemongrass), Azadirachta indica (neem) leaf, Curcuma xanthorrhiza root, Emblica officinalis fruit, Terminalia belerica fruit, Terminalia chebula fruit, and flow and binding agents.

### CROSS REFERENCE TO RELATED APPLICATION

This application claims the benefit of U.S. Provisional Application, Ser. No. 63/465,420, filed on 10 May 2023. The co-pending provisional application is hereby incorporated by reference herein in its entirety and is made a part hereof, including but not limited to those portions which specifically appear hereinafter.

### BACKGROUND OF THE INVENTION

### Field of the Invention

This invention relates generally to an oral treatment composition, such as a toothpowder for cleaning teeth and/or treating dry mouth.

### Description of Prior Art

It is desirable to maintain fresh-tasting mouthfeel throughout the day. Saliva balance is one way to ensure such mouthfeel. Traditional toothpastes may help create such mouthfeel but may fall short in maintaining saliva balance. Traditional toothpastes also fall short in sustainability as hundreds of millions of toothpaste tubes find their way into landfills every year. A need therefore exists for a natural composition that maintains saliva balance and fresh mouthfeel without excessive packaging waste.

### SUMMARY OF THE INVENTION

The invention generally relates to an oral treatment composition, preferably embodied as a toothpowder composition formulated with clean, natural ingredients for holistic care of teeth, gums, breath, saliva, and the oral microbiome.

The subject composition preferably includes organic ayurvedic ingredients like neem, miswak and triphala. The ingredients are preferably compacted into a mouth crushable tab that you bite to crush before brushing. The tabs may be distributed in glass bottles with bamboo tops thereby increasing sustainability of all portions of the product and package.

Embodiments of the invention include an oral treatment composition comprising effective amounts of organic ingredients including neem, miswak, and triphala. The composition can be used as a loose powder, but is preferably embodied as a mouth chewable/breakable tablet. The composition is useful as a toothpowder for cleaning teeth, and the mouth overall. The composition is also useful and effective for reducing or eliminating xerostomia (i.e., “dry mouth”).

In some embodiments, the oral treatment tablet includes effective amounts of an organic powder, desirably pressed into a tablet, including ground neem leaf, ground miswak stem, and ground triphala fruits. As used herein, “triphala” (Sanskrit for “three fruits”) refers to an Ayurvedic herbal rasayana formula consisting of equal parts of three myrobalans, taken without seed: amalaki (also known as Indian gooseberry; e.g., Emblica officinalis), bibhitaki (e.g., Terminalia bellirica), and haritaki (e.g., Terminalia chebula).

In embodiments, the oral treatment composition includes as triphala a combination of amalaki fruit, bibhitaki fruit, and haritaki fruit. The triphala desirably is a combination of powdered Emblica officinalis fruit, Terminalia belerica fruit, and Terminalia chebula fruit. In embodiments, the neem is powdered Azadirachta indica leaf, and the miswak is powdered Salvadora persica stem.

In embodiments, the oral treatment composition includes xylitol and/or bentonite. Xylitol is added to assist in preventing plaque buildup and/or enhancing saliva flow. Bentonite can be beneficial for alkalizing the mouth, remineralizing teeth, polishing teeth, and/or neutralizing bacteria.

In embodiments, the oral treatment composition includes clove and/or lemongrass. Clove is an antiseptic, analgesic, and/or breath freshener. Lemongrass is an antibacterial, is antiplaque, and/or is an antioxidant.

In embodiments, the oral treatment composition includes Curcuma xanthorrhiza root powder. Curcuma xanthorrhiza is antimicrobial and can increase salivary secretion.

In embodiments, the oral treatment composition includes foaming and/or abrasive components, such as calcium carbonate, sodium bicarbonate, and/or guar gum. Other additives or fillers can be used as needed, such as oat fiber, pea starch, bamboo extract, gum arabic, sunflower oil, sunflower lecithin, or combinations thereof.

The invention includes a use of or method of using an amount of the composition in a mouth, such as to clean teeth and/or to treat xerostomia. In preferred embodiments, the use includes steps of: placing a tablet including the composition of embodiments of this invention into your mouth; biting the tablet to crush the tablet and release the composition; adding a little water (e.g., to form a slurry); and brushing teeth and/or gums with the composition.

Other objects and advantages will be apparent to those skilled in the art from the following detailed description.

### BRIEF DESCRIPTION OF THE DRAWINGS

FIG. 1 shows a representative oral treatment tablet according to embodiments of this invention.

FIG. 2 shows a packaging bottle for a plurality of tablets according to FIG. 1.

### DETAILED DESCRIPTION OF THE INVENTION

The present invention provides an oral treatment composition. The composition may include one or more of the following ingredients: xylitol, bentonite, calcium carbonate, sodium bicarbonate, Salvadora persica stem, Cyamopsis tetragonoloba (guar) gum, Eugenia caryophyllus (clove), Cymbopogon flexuosus (lemongrass), Azadirachta indica (neem) leaf, Curcuma xanthorrhiza root, Emblica officinalis fruit, Terminalia belerica fruit, Terminalia chebula fruit, and flow and binding agents.

More specifically, the powder composition of embodiments of this invention may include:
Miswak (Salvadora persica): 2-10% (by weight), more preferably 3-10%-—antibacterial, antifungal, anticariogenic, helps saliva secretion;
Neem (Azadirachta indica): 2-10%, more preferably 2-8%—antibacterial, anticariogenic, oral deodorant, antiplaque, alkalizer;
Triphala (no Latin name as this is a combination of 3 Ayurvedic plants): 2-10%, more preferably 2-8%—antibacterial, antiplaque, anticariogenic, helps strengthen gums and gingiva, helps saliva secretion;
Curcuma xanthorrhiza: 2-10%, more preferably 2-8%—antimicrobial, increases salivary secretion;
Xylitol 20-35%—helps prevent plaque buildup, enhances saliva flow;
Bentonite 10-25%—alkalizes, remineralizes, polishes, helps neutralize bacteria;
Lemongrass (Cymbopogon citratus or Cymbopogon flexuosus) 2-10%, more preferably 2-8%—antibacterial, antiplaque, antioxidant;
Clove (Eugenia caryophyllus): 2-10%, more preferably 2-8%,—antiseptic, analgesic, breath freshener.

Flow and binding ingredients may be selected based on effectiveness of maintaining a tab and disassociating the ingredients during and following use. Traditional agents may be used without affecting the efficacy of the formula, such as magnesium stearate. Flavoring materials, such as spearmint or cardamon, can also be added.

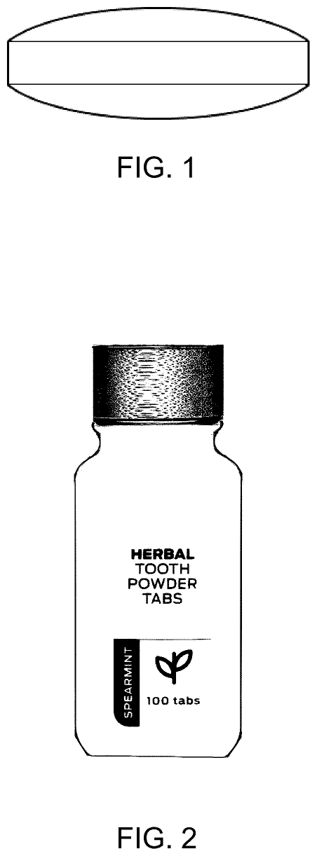

The composition of this invention is preferably formed of powdered and/or extract forms of the above ingredients. The powder composition is formed (e.g., pressed) into a tablet, such as shown in FIG. 1. For use, the tablet is inserted into the mouth and chewed to release the powder from the tablet form. The powder is preferably at least partially dissolvable in the mouth, and then distributed within the mouth, and desirably across the teeth, by a toothbrush or equivalent. FIG. 2 shows a recyclable glass bottle for packaging a plurality of tablets for sale.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. An oral treatment composition comprising effective amounts of organic ingredients including neem, miswak, and triphala.

2. The oral treatment composition according to claim 1, embodied as a mouth chewable tablet.

3. The oral treatment composition according to claim 1, comprising a tooth powder.

4. The oral treatment composition according to claim 1, wherein the composition at least partially treats xerostomia.

5. The oral treatment composition according to claim 1, wherein the triphala comprises a combination of amalaki fruit, bibhitaki fruit, and haritaki fruit.

6. The oral treatment composition according to claim 1, further comprising xylitol and/or bentonite.

7. The oral treatment composition according to claim 1, further comprising clove and/or lemongrass.

8. The oral treatment composition according to claim 1, further comprising Curcuma xanthorrhiza root.

9. The oral treatment composition according to claim 1, further comprising calcium carbonate, sodium bicarbonate, and/or guar gum.

10. The oral treatment composition according to claim 1, wherein the neem comprises Azadirachta indica leaf.

11. The oral treatment composition according to claim 1, wherein the miswak comprises Salvadora persica stem.

12. The oral treatment composition according to claim 1, wherein the triphala comprises Emblica officinalis fruit, Terminalia belerica fruit, and Terminalia chebula fruit.

13. The oral treatment composition according to claim 1, further comprising additives selected from oat fiber, pea starch, bamboo extract, gum arabic, sunflower oil, sunflower lecithin, or combinations thereof.

14. Use of the oral treatment composition according to claim 1 to clean teeth.

15. Use of the oral treatment composition according to claim 1 to treat xerostomia.

16. A method of oral treatment, comprising:
placing a tablet including the composition of claim 1 into a mouth;
biting the tablet to crush the tablet and release the composition; and
brushing teeth and/or gums with the composition.

17. A method of cleaning teeth, comprising applying an amount of the oral treatment composition according to claim 1 to a mouth.

18. An oral treatment tablet comprising effective amounts of an organic powder including ground neem leaf, ground miswak stem, and ground triphala fruits.
